(19) United States
(12) Patent Application Publication
(10) Pub. No.: US 2010/0256058 A1
(43) Pub. Date: Oct. 7, 2010

(54) EXERCISE PERFORMANCE AND RECOVERY FORMULATIONS

(75) Inventor: John Ivy, Austin, TX (US)

Correspondence Address: KNOBBE MARTENS OLSON & BEAR LLP, 2040 MAIN STREET, FOURTEENTH FLOOR, IRVINE, CA, 92614, US

(73) Assignee: GILMORE SPORTS II, LLC, San Marcos, TX (US)

(21) Appl. No.: 12/671,016

(22) PCT Filed: Jul. 25, 2008
(86) PCT No.: PCT/US08/71238

§ 371 Date: Jan. 27, 2010

Related U.S. Application Data
(60) Provisional application No. 60/952,303, filed on Jul. 27, 2007.
(60) Provisional application No. 60/954,060, filed on Aug. 6, 2007.
(60) Provisional application No. 60/970,068, filed on Sep. 5, 2007.
(60) Provisional application No. 60/970,297, filed on Sep. 6, 2007.
(60) Provisional application No. 60/971,852, filed on Sep. 12, 2007.

(51) Int. Cl. A61K 38/16; A61P 3/02

(52) U.S. Cl. 514/5.6

(57) ABSTRACT

Preferred embodiments of the present invention relate to exercise performance and recovery formulations. The exercise performance formulation, according to some embodiments, can be used in sports drinks to enhance endurance and decrease muscle recovery time, thereby improving performance. In other embodiments, the exercise recovery formulation, which can also be used in sports drinks, optimizes recovery from exercise, stimulates muscle glycogen repletion, tissue repair and protein synthesis, and enhances the rate of training adaptation. A dual formulation system comprising both the performance and recovery formulations is also provided.

### CROSS-REFERENCE TO RELATED APPLICATIONS

This application claims the benefit of U.S. Provisional Application Nos. 60/952,303 filed Jul. 27, 2007; 60/954,060 filed Aug. 6, 2007; 60/970,068 filed Sep. 5, 2007; 60/970,297 filed Sep. 6, 2007; and 60/971,852 filed Sep. 12, 2007, all herein incorporated by reference.

### BACKGROUND

1. Field of the Invention

Several preferred embodiments of the present invention relate to exercise performance formulations and exercise recovery formulations. The exercise performance formulation, according to some embodiments, can be used in sports drinks to enhance endurance and decrease muscle recovery time, thereby improving performance. In other embodiments, the exercise recovery formulation, which can also be used in sports drinks, optimizes recovery from exercise, stimulates muscle glycogen repletion, tissue repair and protein synthesis, and enhances the rate of training adaptation.

2. Description of the Related Art

Several exercise drinks are currently on the market. These drinks claim to help individuals, particularly athletes, replenish electrolytes and other nutrients that may be depleted as a result of exercise. However, many of these drinks only provide, at best, an immediate and short-lasting energy boost, accompanied by a subsequent fall in energy levels. Moreover, several drinks contain a high amount of carbohydrates, with no protein, and are high caloric, high glycemic, or both.

### SUMMARY OF THE INVENTION

In several embodiments of the present invention, formulations comprising an optimal carbohydrate-to-protein ratio are provided. According to several embodiments of the present invention, formulations that comprise a unique blend of carbohydrates and protein are provided to enhance exercise performance or recovery. In one embodiment, an exercise performance formulation (or workout formulation) comprises a carbohydrate to protein ratio of about 2.4-2.6:1 (e.g., 2.5:1). In another embodiment, an exercise recovery formulation comprises a carbohydrate to protein ratio of about 2.5-2.7:1 (e.g., 2.6:1).

In one embodiment, the exercise performance and recovery formulations comprise an optimal blend of carbohydrate and protein for better performance and faster recovery. In several embodiments, the formulations comprise whey protein isolate, which is rich in essential amino acids and provides quick muscle recovery. Maltodextrin, a complex carbohydrate for long-lasting, sustained energy, is also included. In some embodiments, the following ingredients are also included: sodium to replenish losses due to perspiration; magnesium to aid in the protein synthesis that helps prevent muscle breakdown; and potassium to help keep body fluids in balance. Additional vitamins and minerals may also be included.

In several embodiments, the exercise performance and recovery formulations are free of caffeine, gluten, and lactose. The formulations, according to several embodiments, are compliant with major sports governing bodies, and are free from substances that are prohibited by these bodies. In several embodiments, the exercise performance and recovery formulations are low in sugar and/or calories, while still providing one or more of the following benefits: reducing muscle damage; enhancing muscle tissue repair and development; replenishing muscle fuel stores rapidly; reducing muscle soreness; increasing rate of fuel absorption; improving fluid retention; and enhancing the hydration process.

In several embodiments, a method of using, or instructing to use, the exercise performance and recovery formulations is provided. In one embodiment, a user is instructed to consume the exercise performance formulation before and during exercise and/or to consume the exercise recovery formulation after completing a workout (e.g., within about 10, 20, 30, 45, 60, 120 minutes after the workout).

In one embodiment, the exercise performance and recovery formulations are complementary and designed to be consumed as part of a program. For example, a user is instructed to consume an exercise performance formulation prior to and during exercise to improve endurance and reduce muscle tissue damage and to subsequently consume the exercise recovery formulation (within, e.g., about 30-45 minutes of completing a workout) to speed the storage of muscle and liver glycogen and promote muscle tissue repair. Thus, in one embodiment, a kit or package comprising both the exercise performance and exercise recovery formulations are provided, along with instructions for use. In other embodiments, a method of affecting physiological factors to improve exercise endurance and reduce muscle tissue damage is provided, wherein said method comprises providing a user with an exercise performance formulation and an exercise recovery formulation, and instructing the user when to consume said formulations (e.g., before/during exercise and within 45 minutes post-exercise). Physiological factors that may be affected include, but are not limited to: muscle glucose uptake, glycogen storage, muscle protein breakdown; cortisol, catecholamines and cytokine levels, insulin levels, muscle soreness, tissue repair, tissue development, fluid retention; hydration, and protein synthesis.

Sports drinks described in U.S. Pat. Nos. 6,207,638 and 6,989,171, hereby incorporated by reference, provide a formulation containing carbohydrates and proteins in a ratio of 2.8 to 4.2 parts of carbohydrate to 1.0 part of proteins. According to U.S. Pat. Nos. 6,207,638 and 6,989,171, a ratio of 4:1 (carbohydrate to protein), provides increased insulin stimulation and enhances the synthesis of muscle glycogen with no negative impact on rehydration following exercise. These references assert that the level of protein is critical, and that “[w]hen the carbohydrate to protein ratio is less than 2.8, the protein has an adverse effect on gastric emptying which would slow rehydration and glucose absorption during exercise.”

Until Applicant's invention, it had been unappreciated that a carbohydrate to protein ratio of less than 2.8 offers several advantages. Despite the express teaching away in the art of using a ratio of less than 2.8, Applicant has discovered that a carbohydrate to protein ratio of less than 2.8 is not only beneficial for both exercise performance and exercise recovery, but offers several benefits and advantages over formulations with higher ratios.

In several embodiments of the invention, a dual formulation system for enhancing one or more physiological effects during both exercise performance and post-exercise recovery is provided. In one embodiment, the dual formulation system comprises an exercise performance formulation and an exercise recovery formulation. The exercise performance formulation comprises a concentration of at least one type of carbohydrate in the range of about 2.4-4.5 g/100 ml and a concentration of at least one type of protein in the range of about 1.1-1.5 g/100 ml. The exercise recovery formulation comprises a concentration of at least one type of carbohydrate in the range of about 10-16 g/100 ml and a concentration of at least one type of protein in the range of about 4-7 g/100 ml. The ratio of carbohydrate to protein in both formulations is about 2.4 to 2.75 (e.g., about 2.5 or 2.6). The dual formulation system further comprises instructions to consume the exercise performance formulation before or during exercise and consuming the exercise recovery formulation within about forty-five minutes post-exercise.

The exercise performance and recovery formulations, used either alone or as part of a dual system, may be provided in liquid, gel or solid form. For example, the formulations may be provided as a sports drink or powder. The formulations, alone or as part of a system, may be used to, in some embodiments, to: (i) regulate muscle glucose uptake and reduce muscle protein breakdown; (ii) suppress cortisol, catecholamines and cytokine levels; and/or (iii) enhance the rate of protein synthesis and facilitate training adaptation.

### DETAILED DESCRIPTION OF EMBODIMENTS

### Exercise Performance (Workout) Formulation

In one preferred embodiment, an exercise performance (or workout) formulation is provided. In one embodiment, a formulation comprising a ratio of carbohydrate to protein of less than 2.8:1 is provided. In one embodiment, the formulation is prepared for use as a supplement. In one preferred embodiment, the formulation is provided as an energy or sports drink.

In one embodiment, the exercise performance formulation optimizes aerobic exercise performance and reduces muscle damage during exercise. The formulation, in other embodiments, also enhances rehydration post exercise. Contrary to teachings in the related art, preferred embodiments of the present invention do not have an adverse effect on gastric emptying, and do not slow rehydration and glucose absorption during exercise. Indeed, preferred embodiments of the present invention facilitate rehydration associated with exercise and facilitate glucose absorption.

Exercise results in dehydration and the development of a catabolic state in which the body recruits its endogenous fuel sources for energy resulting in the depletion of muscle and liver glycogen and the breakdown of muscle protein. During this catabolic state there is also suppression of the immune system. Preferred embodiments of the present invention provide an exogenous source of carbohydrate, reduce the reliance on endogenous fuel sources, and extend time to exhaustion during aerobic exercise despite a low caloric content. In addition, the protein contained in preferred embodiments will reduce damage to muscle tissue. In one embodiment, the drink will also reduce the risk of dehydration, enhance rehydration, and help support the immune system.

In one embodiment, the exercise performance formulation comprises a unique blend of carbohydrates along with a whey protein isolate. In one embodiment, this combination stimulates two cellular signaling pathways that regulate muscle glucose uptake and reduce muscle protein breakdown. In one embodiment, the blend of carbohydrates will increase the rate of glucose uptake and, in combination with the protein, will increase the blood insulin response above that of carbohydrate alone. This will result in muscle glycogen sparing and inhibition of muscle protein breakdown. The high insulin response coupled with the increase in blood glucose post exercise will also suppress blood cortisol, catecholamines and cytokine levels, and reduce stress on the immune system. Moreover, the addition of protein to the drink will help with fluid retention and rehydration post exercise. Several embodiments of the present invention, which comprise a ratio of carbohydrate to protein in a ratio of less than 2:8:1 (e.g., a ration of 2.5:1), offer specific advantages because of the additive or synergistic effects of the carbohydrates and proteins in the given ratio.

In one embodiment, the exercise performance formulation can be taken before, during, and/or after exercise. The formulation, according to several preferred embodiments, increases the rate of glucose uptake, providing an exogenous carbohydrate fuel source, and provides amino acids, which help to reduce muscle damage during exercise. In one embodiment, the carbohydrate and protein work additively or synergistically to promote better fuel utilization. The formulation, according to preferred embodiments, also supports the immune system. The electrolytes provide replacement electrolytes lost during exercise and, when combined with the protein, will increase fluid retention more effectively than a carbohydrate/electrolyte formulation that lacks protein.

As discussed earlier, the ratio of carbohydrate to protein in preferred embodiments of the present invention is less than 2.8:1. In some embodiments, the ratio is in the range of 1.85 to 2.75. In other embodiments, the ratio is in the range of 1.0 to 1.85. In yet other embodiments, the ratio is 2.4, 2.5, 2.6, or 2.7.

In one embodiment, the exercise performance formulation of the present invention comprises the following ingredients, in the following approximate concentrations:

| | |
|---|---|
| Dextrose | 0.8-1.5 g/100 ml |
| Maltodextrin | 0.8-1.5 g/100 ml |
| Fructose | 0.8-1.5 g/100 ml |
| Whey Protein | 1.1-1.5 g/100 ml |
| Sodium | 70 mg/100 ml |
| Magnesium | 30 mg/100 ml |
| Potassium | 30 mg/100 ml |
| Vitamin C | 15 mg/100 ml |
| Water | 100 ml |

In other embodiments, the following concentrations are used:

| | |
|---|---|
| Carbohydrates | 1-8.25 g/100 ml |
| Protein | 1-3 g/100 ml |
| Sodium | 0-100 mg/100 ml |
| Magnesium | 0-100 mg/100 ml |
| Potassium | 0-100 mg/100 ml |
| Vitamin C | 0-1000 mg/100 ml |
| Water | 100 ml |

In one embodiment, the exercise performance formulation comprises dextrose, maltodextrin, and fructose as the preferred carbohydrate source. In one embodiment, maltodextrin is advantageous because it reduces the osmolality of the drink, thereby permitting faster gastric emptying. The dextrose and maltodextrin breakdown in the intestines and are transported into the circulatory system as glucose by glucose transporters. Fructose is transported by its own transporter, thus increasing the rate of carbohydrate uptake and producing a greater reliance on exogenous fuel during exercise.

In one embodiment, the exercise performance formulation comprises the following ingredients in an approximately 80 calorie serving: fat (0 g); sodium (330 mg); potassium (140 mg); carbohydrate (15 g); protein (6 g); vitamin C (110% of the daily recommended intake); and magnesium (35% of the daily recommended intake).

In one embodiment, the exercise performance formulation comprises whey protein, dextrose, maltodextrin, crystalline fructose, citric acid, sodium chloride, magnesium sulfate, monopotassium phosphate, natural and artificial flavors, silicon dioxide, ascorbic acid, sucralose, yellow 5 lake, and blue 1 lake.

Although certain preferred embodiments of the invention comprise a formulation for exercise performance, certain embodiments will be used for rehydration therapy in conditions or illnesses associated with fluid loss, including, but not limited to, dysentery.

### Exercise Recovery Drink

In one preferred embodiment, an exercise recovery drink is provided. In one embodiment, an energy formulation comprising a ratio of carbohydrate to protein of less than 2.8:1 is provided. In one embodiment, the formulation is prepared for use as a supplement. In one preferred embodiment, the formulation is provided as an energy or sports drink.

In one embodiment, the exercise recovery formulation provides one or more of the following advantages: optimizes recovery from exercise, stimulates muscle glycogen repletion, tissue repair and protein synthesis, and enhances the rate of training adaptation. In a preferred embodiment, the exercise recovery formulation provides all three of the advantages identified above. As discussed earlier, exercise results in the development of a catabolic state in which the body recruits its endogenous fuel sources for energy resulting in the depletion of muscle and liver glycogen and the breakdown of muscle protein. During this catabolic state, suppression of the immune system also occurs. Post exercise, the consumption of the exercise recovery drink will rapidly provide carbohydrates for the replenishment of muscle and liver glycogen and amino acids for the repair of muscle. Moreover, according to one embodiment, the formulation will result in a cellular environment that: (i) enhances the rate of protein synthesis, (ii) facilitates faster training adaptation, and (iii) supports the immune system.

In one embodiment, the exercise recovery formulation of the present invention comprises a unique blend of carbohydrates along with a whey protein isolate. In one embodiment, this combination stimulates two cellular signaling pathways that regulate muscle glycogen storage and protein synthesis. In one embodiment, the blend of carbohydrates will increase the rate of glucose uptake and, in combination with the protein, will increase the blood insulin response above that of carbohydrates alone. In combination with an enhanced activation of the insulin signaling pathway, rapid uptake of carbohydrate and elevation in plasma insulin, as well as muscle and liver glycogen storage, are rapidly increased. Likewise, the elevation in insulin and blood amino acids from the ingestion of the protein will act additively or synergistically on the mTorr-signaling pathway. This will increase the rate of mRNA translation and protein synthesis, promoting muscle tissue repair and stimulate training adaptation, which is the over expression of proteins involved in exercise performance. In one embodiment, the high insulin response coupled with the increase in blood glucose post exercise will also suppress blood cortisol, catecholamines and cytokine levels and reduce stress on the immune system.

Preferred embodiments of the exercise recovery formulation, which comprise a ratio of carbohydrate to protein in a ratio of less than 2:8:1, offer several advantages because of the combined effects of the carbohydrates and proteins in the given ratio. In some embodiments, the carbohydrates and proteins produce additive results. In other embodiments, the carbohydrates and proteins produce synergistic results.

As discussed earlier, the ratio of carbohydrate to protein in embodiments of the exercise recovery formulation is less than 2.8:1. In some embodiments, the ratio is in the range of 1.85 to 2.75. In other embodiments, the ratio is in the range of 1.0 to 1.85. In yet other embodiments, the ratio of the recovery formulation is 2.4, 2.5, 2.6, or 2.7.

In one preferred embodiment, the exercise recovery formulation of the present invention comprises the following ingredients, in the following approximate concentrations:

| | |
|---|---|
| Dextrose | 4-6 g/100 ml |
| Maltodextrin | 4-6 g/100 ml |
| Fructose | 2-4 g/100 ml |
| Whey Protein | 4-7 g/100 ml |
| Sodium | 70 mg/100 ml |
| Magnesium | 80 mg/100 ml |
| Potassium | 30 mg/100 ml |
| Vitamin C | 90 mg/100 ml |
| Water | 100 ml |

In other embodiments, the following concentrations are used:

| | |
|---|---|
| Carbohydrates | 5-30 g/100 ml |
| Protein | 2-15 g/100 ml |
| Sodium | 0-100 mg/100 ml |
| Magnesium | 0-100 mg/100 ml |
| Potassium | 0-100 mg/100 ml |
| Vitamin C | 0-1000 mg/100 ml |
| Water | 100 ml |

In one embodiment, the exercise recovery formulation comprises the following ingredients in an approximately 260 calorie serving: fat (0 g); cholesterol (5 mg) sodium (250 mg); potassium (125 mg); carbohydrate (47 g); protein (18 g); vitamin C (520% of the daily recommended intake); and iron (2% of the daily recommended intake).

In one embodiment, the exercise recovery formulation comprises whey protein, dextrose, maltodextrin, crystalline fructose, citric acid, magnesium sulfate, natural and artificial flavors, silicon dioxide, ascorbic acid, monopotassium phosphate, sodium chloride, sucralose, yellow 5 lake, and blue 1 lake.

In one embodiment, the exercise recovery drink can be taken before, during, and/or after exercise. In preferred embodiments, the formulation increases the rate of glucose uptake, providing carbohydrate for glycogen replenishment, and provides amino acids for protein synthesis. According to some embodiments, the carbohydrate and protein work additively or synergistically to promote glycogen storage, protein synthesis and muscle tissue repair. The formulation of some embodiments also support the immune system. The electrolytes provided replace electrolytes lost during exercise, but also increase glucose uptake from the intestines to the circulation.

### Exercise Performance And Recovery Formulations

Both the exercise performance and recovery formulations described herein comprise a ratio of about 1-2.75 carbohydrate for every 1 protein. Preferably, the ratio is 1.85 to 2.75. In other embodiments, the ratio of carbohydrate to protein is 1.0, 1.2, 1.4, 1.6, 1.8, 2, 2.2, 2.4, or 2.6. In one embodiment, the performance formulation has a ratio of 2.4-2.6 and the recovery formulation has a ratio of 2.5-2.7.

Carbohydrates used in several embodiments of the exercise performance and recovery formulations include dextrose, maltodextrin, and fructose, but may include other carbohydrates. For example, in some embodiments, one or more of the following carbohydrates can be used instead of or in addition to dextrose, maltodextrin, and/or fructose: glucose, maltose, maltotriose, lactose, galactose, sucrose, high fructose corn syrup, beet sugar, cane sugar, arabinose, ribose, sorbose, tagatose and sorbitol.

In several embodiments, a combination of different carbohydrates (rather than a single type) is used to increase the rate of muscle fuel (glycogen) reloading and recovery. In some embodiments, the combination of carbohydrates used for both the performance and recovery formulations is dextrose, maltodextrin, and fructose. Using a blend of carbohydrates provides, in some embodiments, a beneficial diversity in absorption rates and glycemic index, which in turn can enhance the rate of glycogen reloading and recovery.

Sweetening agents may also be used in several embodiments. The sweetening agents may be a source of carbohydrate (such as glucose or fructose), or may be an artificial sweetener with negligible caloric value, including but not limited to, sucralose, stevia, saccharin, and aspartame. Other flavoring agents may also be used. Caffeine or other stimulants may be included in alternative embodiments. Epigallocatechin gallate (EGCG), may be used in some embodiments. In several embodiments, the formulations do not contain any caffeine, EGCG, or like stimulant.

In one embodiment, the exercise performance and recovery formulations comprise whey protein (isolate, hydrolyzate, or concentrate) as the preferred protein source. The whey protein contains all essential amino acids in high concentration and readily dissolves in solution when mixed. Proteins other than whey may also be used. For example, one or more of the following proteins can be used instead of or in addition to whey protein: soy protein, egg protein, rice protein, casein, and protein blends. Individual amino acids may also be used.

In one embodiment, the exercise performance and recovery formulations comprise sodium, magnesium, and potassium to replace electrolytes lost during exercise. Electrolytes that may be used instead of or in addition to sodium, magnesium, and/or potassium include, but are not limited to, calcium. Other electrolytes may also be used.

In one embodiment, the exercise performance and recovery formulations comprise Vitamin C as an antioxidant. Other antioxidants may also be used, including but not limited to vitamin A, beta carotene, and alpha lipoic acid. In some embodiments, other vitamins and minerals are included. For example, vitamin B may be used in some embodiments.

In one embodiment, the exercise performance and recovery formulations are provided in a powdered or otherwise dehydrated form. In one embodiment, instructions for use that instruct a user to add water (or other liquid) to the dehydrated form are provided. In other embodiments, the formulations are provided in a sustained or slow release form. In some embodiments, the formulations are provided in solid or gelatinous forms. In yet other embodiments, the formulations are provided in a soft chew, hard candy, or gum format.

In one embodiment, the exercise performance and recovery formulations are free from lactose, and are therefore suitable for individuals that are lactose intolerant. In several embodiments, the formulations are gluten free. In other embodiments, the formulations are caffeine free. In yet other embodiments, the formulations are free from any compound prohibited by governing sports authorities.

In one embodiment, the exercise performance and recovery formulations comprise or consist essentially of certified organic ingredients or other natural ingredients.

Unlike the drinks disclosed in the related art, several embodiments of the present invention provide an exercise performance and/or recovery drink that is low in calories, while still enhancing performance and recovery. In drinks that use a 4:1 ratio of carbohydrate to protein, 20 grams of carbohydrates would be needed in any drink that contained just 5 grams of protein. This high quantity of carbohydrates is unpalatable to many recreational and professional athletes. By contrast, in embodiments of the present invention, an exercise drink can contain 5 grams of protein, and only 5 grams to 13.75 grams of carbohydrates. In one embodiment, the reduction in carbohydrates allows a sports drink to contain fewer calories. In another embodiment, the caloric value can be the same as other drinks, but more of those calories can come from protein instead of carbohydrates. Lower caloric values per serving size, according to some embodiments of the formulations, are particularly advantageous in terms of the reduced glycemic load of the formulations (e.g., drinks) as compared to other sports or energy drinks or products.

Prior to Applicant's invention, several references taught that “carbo-loading” prior to exercise was the most efficient way to stock muscles with fuel. However, Applicant determined that insulin in the blood was more effective at carrying energy into the muscles if those muscles had recently been active. It is believed that exercise makes muscles more responsive to insulin, and this insulin, in turn, increases glycogen muscle uptake. Exercise prompts muscles to absorb more fuel from the bloodstream. This improved insulin response, however, lasts only for a brief time (e.g., about 30-45 minutes) after a workout. After that, it is believed that muscles become resistant to insulin and much less able to absorb glucose. Thus, consuming carbohydrates within about 30 or 45 minutes after a strenuous workout is beneficial to restoring the glycogen burned. It is believed that waiting a few hours post-workout diminishes the body's ability to restore glycogen by nearly 50%. However, the consumption of carbohydrates alone is not optimal. Protein is also important, and more particularly, the ratio between the carbohydrate and the protein has an significant impact on both performance and recovery. Consuming protein with carbohydrates can accelerate muscle glycogen repletion by stimulating endogenous insulin release. Moreover, specific ratios of protein and carbohydrates, such as those described herein, are more beneficial than others.

In one embodiment, a method of using, or instructing to use, the exercise performance and recovery formulations is provided. For example, in one embodiment, a user is instructed to consume the exercise performance formulation before and during exercise. In another embodiment, a user is instructed to consume the exercise recovery formulation after completing a workout (e.g., within about 10, 20, 30, 45, 60, or 120 minutes after the workout). In some embodiments, the user may be instructed to consume the recovery formulation later than about two hours post-workout. In some preferred embodiments, a user is instructed to consume the recovery formulation within about 30-45 minutes post-exercise.

In one embodiment, the exercise performance and recovery formulations are complementary and designed to be consumed: (i) before and during exercise to improve endurance and reduce muscle tissue damage (with respect to the exercise performance formulation); and (ii) within about 30-45 minutes of completing a workout to speed the storage of muscle and liver glycogen and promote muscle tissue repair (with respect to the exercise performance formulation). Thus, in one embodiment, both the exercise performance and exercise recovery formulations are provided together, along with instructions for use.

It is believed that during exercise, the muscles become sensitive to certain hormones and nutrients, and many highly desirable training adaptations can be initiated if the correct nutrients, in the correct ratios, are present. The increased sensitivity of the muscles typically lasts for a limited length of time; therefore, timing becomes important. Thus, instructing a user to consume the exercise performance formulation before/during a work-out and the exercise recovery formulation post-workout is advantageous in several embodiments because it optimizes the stimulation of certain muscle adaptations.

### Example 1

The following example illustrates non-limiting examples of some embodiments of the invention. To evaluate the advantages of the formulations of certain embodiments of the invention, a study comparing the effects of a formulation having a carbohydrate to protein ratio of 2.5:1 with formulations having either a 4.2:1 ratio or a carbohydrate-only formulation.

In the study, the “2.5 ratio” drink comprised a 3% carbohydrate blend (maltodextrin, dextrose and fructose) with 1.2% whey protein. The “4.2 ratio” drink comprised a 5.9% carbohydrate blend with 1.4% protein. The “Carb-Only” drink comprised 6% carbohydrate. The “2.5 ratio” drink was lower in calories than the other two drinks.

The subjects tested included 10 competitive cyclists who are accustomed to cycling for prolonged periods (3-5 h). They were between the ages of 19 and 33 years old. Each subject completed three randomly assigned treatments in which either the “Carb-Only”, the “2.5 ratio” drink, or the “4.2 ratio” drink was provided during exercise. At the beginning of exercise and every 20 min thereafter, 300 ml of the drink was provided. The subjects performed each trial in a room of 19-21° C. at the same time of the day and the same day of the week over a 3-week period.

Subjects reported to the laboratory before the start of the experiment for determination of their VO₂ max and a practice trial. The VO₂ max test was performed on the same ergometer used in the experimental trials (Ergometrics 800-S, Sensormedics, Ergo-line GmbH, Bitz, Germany). The protocol for establishing VO₂ max consisted of a 4 min warm up and then 2 min stages beginning at 200 watts (W) and increasing the workload with 50 W every 2 min until 350 W. After 350 W the increase was 25 W every minute. Subjects breathed through a Daniel's valve, with expired gases directed to a mixing chamber for analysis of oxygen (O₂) and carbon dioxide (CO₂). Inspired volumes were measured using a dry gas meter (Max-1, Physio-Dyne Instruments corp., Quogue, N.Y.). Outputs from these instruments were directed to a laboratory computer for calculation of ventilation, O₂ consumption (VO₂), CO₂ production, and respiratory exchange ratio (RER) every 30 1. The criteria used to establish VO₂ max were a plateau in VO₂ with increasing exercise intensity and RER>1.10.

Two to three days after the VO₂ max test the subjects reported to the laboratory to perform a practice ride to adjust and/or verify appropriate workloads for the experimental trials. No blood samples were collected during the practice ride. The practice ride consisted of cycling at variable intensities between 45% and 70% VO₂ max for 3 h followed by cycling at 80% VO₂ max until fatigue. During the ride the subjects received 300 ml of water every 20 min. They were encouraged to ride as long as possible while maintaining a peddling cadence of 80 to 90 revolutions per minute (rpm). When the subject could longer maintain a peddling cadence of 65 rpm they were asked to stop. During the exercise trial, the laboratory temperature was maintained at 19-21° C. and two floor fans directed towards the subject to reduce thermal stress. The subjects were instructed to maintain a training and dietary log for two days before the first experimental trial. The subjects provide a photocopy of their training and dietary log and were instructed to have the same dietary intake and activity the 48 h before each trial. Each experimental trial was separated by a minimum of 7 days.

The subjects reported to the lab in the morning after a 12 h fast during which time they were allowed to consume only water. On reporting to the laboratory, body weight was obtained and a heart rate monitor (Polar Beat, Polar Electro Oy, Finland) secured in place around their chest. A catheter was inserted into a forearm vein, fitted with a three-way stopcock, extended with a catheter-extension, and taped in place. The subjects then mount the ergometer and performed the above-described protocol used for the practice trial. Supplements (300 ml) were provided every 20 min, and respiratory gas samples were collected between 5-10 min and then at every 30 min of exercise for 5 min.

The subjects and the investigators were unaware as to whether the subjects were on the “Carb-Only”, the “2.5 ratio” drink, or the “4.2 ratio” treatment. The three treatments (drinks) were similar in color, taste, and texture and were randomly distributed by a laboratory technician not involved in the data collection. Constant verbal encouragement was given to the subjects during each trial. During each ride they were not aware of how long they have ridden, as all timing devices were removed from the sight of the subjects

Ventilation, VO₂, CO₂ production, and RER were recorded with the respiratory gas analysis system described previously. Respiratory gas collection periods were limited to 5 min but only the last 3 min for each collection were used to determine VO₂ and RER. Heart rate (HR) was recorded at the beginning of exercise and at every 30 min of exercise. Subjective ratings of perceived exertion on a Borg-scale (ranging from 6 to 20) were obtained during exercise at the same time points as heart rate. Five milliliters of venous blood was drawn while the subjects were seated on the cycle ergometer immediately before the start of exercise, during exercise at every 30 min of exercise, and immediately after exercise. One drop of blood was used to measure plasma glucose concentration with a glucose meter (One Touch Basic, Lifescan Inc., Milpitas, Calif., USA). Each blood sample was anticoagulated with 0.3 ml of EDTA (24 mg/ml, pH 7.4). 0.5 ml of the anticoagulated blood and then transferred to another tube containing 1 ml 10% perchloric acid. All tubes were centrifuged for 15 min at 3,000 rpm with a JS-7.5 rotor in a Beckman J2-21 centrifuge. Respectively, plasma and perchloric acid extracts were transferred and stored at −80° C. for further analysis. The plasma samples will be analyzed for insulin and myoglobin by radioimmunoassay. Blood lactate will be determined from the perchloric extract by enzymatic analysis.

Initial results from 10 of 12 for physical performance were as follows: The average time (minutes) in which the subjects were able to ride at the highest workload for each treatment was:

“2.5 ratio” drink: 25.04±6.1 minutes;
“Carb-Only” drink: 21.17±3.3 minutes; and
“4.2 ratio” drink: 18.75±2.8 minutes.

Accordingly, with the “2.5 ratio” drink, which was prepared in accordance with several embodiments of the present invention, data suggest that physical performance was about 18% better than the “Carb-Only” drink, and 33% better with the “4.2 ratio” drink.

The data further revealed that 6 of the 10 subjects exhibited superior performance when provided with the “2.5 ratio” drink. For example, the average times to fatigue were as follows:

“2.5 ratio” drink: 35.0 minutes;
“Carb-Only” drink: 25 2 minutes; and
“4.2 ratio” drink: 22.7 minutes.

Accordingly, with the “2.5 ratio” drink, which was prepared in accordance with several embodiments of the present invention, physical performance (in terms of increased time to fatigue) was enhanced.

Therefore, the data from the preliminary study discussed above suggest that a “2.5 ratio” drink in accordance with several embodiments of the present invention (e.g., the exercise performance formulations) offers benefits over other types of sports drinks. Moreover, these benefits occur despite the “2.5 ratio” drink having fewer calories than the other two drinks. For example, the “2.5 ratio” drink comprised a 3% carbohydrate blend with 1.2% protein, which amounts to about 84 calories per 500 ml. The “4.2 ratio” drink comprised a 5.9% carbohydrate blend with 1.4% protein, which amounts to about 146 calories per 500 ml. The “Carb-Only” drink comprised 6% carbohydrate, which amounts to about 146 calories per 500 ml. Thus, in accordance with several embodiments of the invention, the “2.5 ratio” drink offers enhanced performance with fewer calories. The reduced caloric value and/or the reduced sugar content of the “2.5 ratio” drink are particular advantages of several embodiments of the invention.

It will be understood by those of skill in the art that numerous and various modifications can be made without departing from the spirit of the present invention. Therefore, it should be clearly understood that the forms of the present invention are illustrative only and are not intended to limit the scope of the present invention.

1.-16. (canceled)

17. An exercise performance formulation comprising:
at least three different types of carbohydrates, wherein the total concentration of said carbohydrates is in the range of about 2.4-4.5 g/100 ml,
wherein said carbohydrates comprise dextrose, maltodextrin, and fructose;
at least one protein in the range of about 1.1-1.5 g/100 ml,
wherein said protein comprises whey protein;
wherein the ratio of said carbohydrates to said protein in said exercise performance formulation is in the range of 2.4 to 2.6.

18. The exercise performance formulation of claim 17,
wherein said exercise performance formulation is free from caffeine, lactose, and gluten.

19. The exercise performance formulation according to claim 17,
wherein said dextrose has a concentration in the range of about 0.8-1.5 g/100 ml;
wherein said maltodextrin has a concentration in the range of about 0.8-1.5 g/100 ml; and
wherein said fructose has a concentration in the range of about 0.8-1.5 g/100 ml.

20. The exercise performance formulation according to claim 17, further comprising
sodium having a concentration of about 70 mg/100 ml;
magnesium having a concentration of about 30 mg/100 ml;
potassium having a concentration of about 30 mg/100 ml;
vitamin C having a concentration of about 15 mg/100 ml; and
about 100 ml water.

21. The exercise performance formulation according claim 17, wherein said formulation is provided as a powder.

22. The exercise performance formulation of claim 17, wherein consumption of the formulation before or during exercise regulates muscle glucose uptake and reduces muscle protein breakdown.

23. The exercise performance formulation of claim 17, wherein consumption of the formulation before or during exercise suppresses one or more of cortisol release, catecholamine release, and cytokine release.

24. The exercise performance formulation of claim 17, wherein consumption of the formulation before or during exercise enhances the rate of protein synthesis and the rate of training adaptation.

25. The exercise performance formulation of claim 17, wherein consumption of the formulation before or during exercise increases the time to exhaustion during exercise.

26. A kit comprising the exercise performance formulation of claim 17, further comprising an exercise recovery formulation,
wherein said exercise recovery formulation comprises a concentration of at least one type of carbohydrate in the range of about 10-16 g/100 ml,
wherein said carbohydrate in said exercise recovery formulation comprises dextrose, maltodextrin, and fructose,
a concentration of at least one type of protein in the range of about 4-7 g/100 ml,
wherein said protein in said exercise recovery formulation comprises whey protein, and
wherein the ratio of said carbohydrate to said protein in said exercise recovery formulation is in the range of 2.5 to 2.7.

27. An exercise recovery formulation comprising:
at least three different types of carbohydrates, wherein the total concentration of said carbohydrate is in the range of about 10-16 g/100 ml,
wherein said carbohydrates comprise dextrose, maltodextrin, and fructose;
at least one protein in the range of about 4-7 g/100 ml,
wherein said protein comprises whey protein;
wherein the ratio of said carbohydrates to said protein in said exercise recovery formulation is in the range of 2.5 to 2.7.

28. The exercise recovery formulation of claim 27,
wherein said exercise recovery formulation is free from caffeine, lactose, and gluten.

29. The exercise recovery formulation of claim 27,
wherein said dextrose has a concentration in the range of about 4-6 g/100 ml;
wherein said maltodextrin has a concentration in the range of about 4-6 g/100 ml; and
wherein said fructose has a concentration in the range of about 4-6 g/100 ml.

30. The exercise recovery formulation according to claim 27, further comprising:
sodium having a concentration of about 70 mg/100 ml;
magnesium having a concentration of about 80 mg/100 ml;
potassium having a concentration of about 30 mg/100 ml;
vitamin C having a concentration of about 90 mg/100 ml;
iron having a concentration of about 2% of daily recommended intake; and
about 100 ml water.

31. The exercise recovery formulation according to claim 27, wherein said formulation is provided as a powder.

32. The exercise recovery formulation according to claim 27, wherein consumption of the formulation within about forty-five minutes post-exercise regulates muscle glucose uptake and reduces muscle protein breakdown.

33. The exercise recovery formulation according to claim 27, wherein consumption of the formulation within about forty-five minutes post-exercise suppresses one or more of cortisol release, catecholamine release, and cytokine release.

34. The exercise recovery formulation according to claim 27, wherein consumption of the formulation within about forty-five minutes post-exercise enhances the rate of protein synthesis and facilitates training adaptation.

35. A kit comprising the exercise recovery formulation of claim 27, further comprising an exercise performance formulation, wherein the exercise performance formulation comprises a ratio of carbohydrates to protein in a range of 2.4 to 2.6.

36. A method of affecting physiological factors to improve exercise endurance and reduce muscle tissue damage, comprising:
providing an exercise performance formulation to a user with instructions to consume said performance formulation before and/or during exercise;
wherein said exercise performance formulation comprises a concentration of at least one type of carbohydrate in the range of about 2.4-4.5 g/100 ml, and a concentration of at least one type of protein in the range of about 1.1-1.5 g/100 ml,
wherein the ratio of said carbohydrate to said protein in said exercise performance formulation is in the range of 2.4 to 2.75; and
providing an exercise recovery formulation to a user with instructions to consume said recovery formulation within about 45 minutes post-exercise;
wherein said exercise recovery formulation comprises a concentration of at least one type of carbohydrate in the range of about 10-16 g/100 ml, and a concentration of at least one type of protein in the range of about 4-7 g/100 ml,
wherein the ratio of said carbohydrate to said protein in said exercise recovery formulation is in the range of 2.4 to 2.75.
